SEQUENCE LISTING (13 sequences)

SEQ ID NO: 1 - DNA, 2 bp, Artificial sequence
  Synthetic construct
yg

SEQ ID NO: 2 - DNA, 3 bp, Artificial sequence
  Synthetic construct
  misc_feature (1)..(1): n is a, c, g, or t
ngg

SEQ ID NO: 3 - DNA, 3 bp, Artificial Sequence
  Synthetic construct
  misc_feature (1)..(1): n is a, c, g, or t
nga

SEQ ID NO: 4 - DNA, 4 bp, Artificial sequence
  Synthetic construct
  misc_feature (1)..(1): n is a, c, g, or t
ngcg

SEQ ID NO: 5 - DNA, 4 bp, Artificial sequence
  Synthetic construct
  misc_feature (1)..(1): n is a, c, g, or t
ngag

SEQ ID NO: 6 - DNA, 5 bp, Artificial sequence
  Synthetic construct
  misc_feature (1)..(1): n is a, c, g, or t
  misc_feature (4)..(4): n is a, c, g, or t
nggng

SEQ ID NO: 7 - DNA, 6 bp, Artificial sequence
  Synthetic construct
  misc_feature (1)..(2): n is a, c, g, or t
nngrrt

SEQ ID NO: 8 - DNA, 6 bp, Artificial sequence
  Synthetic construct
  misc_feature (1)..(3): n is a, c, g, or t
nnnrrt

SEQ ID NO: 9 - DNA, 6 bp, Artificial sequence
  Synthetic construct
  misc_feature (1)..(1): n is a, c, g, or t
naaaac

SEQ ID NO: 10 - DNA, 8 bp, Artificial sequence
  Synthetic construct
  misc_feature (1)..(4): n is a, c, g, or t
  misc_feature (6)..(7): n is a, c, g, or t
nnnngnnt

SEQ ID NO: 11 - DNA, 7 bp, Artificial sequence
  Synthetic construct
  misc_feature (1)..(2): n is a, c, g, or t
nnagaaw

SEQ ID NO: 12 - DNA, 8 bp, Artificial sequence
  Synthetic construct
  misc_feature (1)..(4): n is a, c, g, or t
  misc_feature (6)..(6): n is a, c, g, or t
nnnncndd

SEQ ID NO: 13 - DNA, 8 bp, Artificial sequence
  Synthetic construct
  misc_feature (1)..(4): n is a, c, g, or t
nnnnryac

(19) United States
(12) Patent Application Publication
(10) Pub. No.: US 2024/0206439 A1
(43) Pub. Date: Jun. 27, 2024

(54) COMPOSITIONS AND METHODS FOR RAPID GENERATION OF MODIFIABLE STABLE CELL LINES

(71) Applicant: Duke University, Durham, NC (US)

(72) Inventors: Scott SODERLING, Durham, NC (US); Akiyoshi UEZU, Durham, NC (US)

(73) Assignee: Duke University, Durham, NC (US)

(21) Appl. No.: 18/288,505

(22) PCT Filed: Apr. 26, 2022
(86) PCT No.: PCT/US2022/026352

§ 371 Date: Oct. 26, 2023

Related U.S. Application Data
(60) Provisional application No. 63/179,585, filed on Apr. 26, 2021.

(51) Int. Cl. A01K 67/0275; C12N 5/0735; C12N 5/074; C12N 9/22; C12N 15/85; C12N 15/90

(52) U.S. Cl. CPC A01K 67/0275; C12N 5/0606; C12N 5/0696; C12N 9/22; C12N 15/85; C12N 15/907; A01K 2217/07; C12N 2800/30

(57) ABSTRACT

Provided herein are methods for generating modifiable, stable cell lines.

### PRIOR RELATED APPLICATION

This application claims the benefit of and priority to U.S. Provisional Application No. 63/179,585 filed on Apr. 26, 2021, which is hereby incorporated by reference in its entirety.

### STATEMENT REGARDING FEDERALLY FUNDED RESEARCH

This invention was made with government support under Grant No. MH111684 awarded by the National Institutes of Health/National Institute of Mental Health. The government has certain rights in the invention.

### FIELD

This disclosure describes compositions and methods for making stable cell lines.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 1308859_seglist.txt, created on Apr. 26, 2022, having a size of 4 kb, and is filed concurrently with the specification. The sequence listing contained in this ASCII formatted document is part of the specification and is herein incorporated by reference in its entirety.

### BACKGROUND

Stable cell line generation methods are used to produce genetically altered cells for research, therapeutic cell-based applications, and the production of biologics. Often, multiple lines with different alterations to the same gene are needed. These approaches are tedious and time-consuming, as each cell line must be carefully characterized to ensure the desired genetic modification has been achieved without off-target effects elsewhere in the genome. This is especially true when utilizing inducible pluripotent stem cell (iPSCs), where isogenic cell lines are often desired. Compositions and methods for rapid generation of stable cell lines are necessary.

### SUMMARY

Provided herein are methods of generating stable cell lines. The methods comprise (a) inserting a nucleic acid sequence encoding a first donor polypeptide into the genome of a population of cells via Homology-independent Universal Genome Engineering (HiUGE), wherein the nucleic acid encoding the first donor polypeptide is flanked on each side by one or more recombinase target sites; (b) selecting cells that express the first donor polypeptide; and (c) exchanging the nucleic acid encoding the first donor polypeptide in the genome of the selected cells with a nucleic acid encoding a second donor polypeptide by contacting the selected cells with: (i) a vector comprising a nucleic acid sequence encoding the second donor polypeptide, wherein the nucleic acid encoding the second donor polypeptide is flanked on each side by the one or more recombinase target sites, and wherein the one or more recombinase target sites are in frame with the coding sequence of the second donor polypeptide; and (ii) a vector encoding a recombinase that cleaves the one or more recombination target sites inserted into the genome of the selected cells and the one or more recombinase target sites in the vector, whereby the nucleic acid encoding the first donor polypeptide is exchanged for the nucleic acid encoding the second donor polypeptide in the genome of the cells via recombination-mediated cassette exchange (RMCE).

In some embodiments, the nucleic acid encoding the second donor polypeptide is inserted into a coding sequence for a gene. In some embodiments, the cell expresses a fusion polypeptide comprising the second donor polypeptide and an endogenous polypeptide encoded by the gene. In some embodiments, the expression of the second donor polypeptide or the fusion polypeptide is under the control of the endogenous promoter for the gene.

In some embodiments, the vector comprising the nucleic acid sequence encoding the second donor polypeptide further comprises an exogenous promoter operably linked to the nucleic acid sequence encoding the second donor polypeptide. In some embodiments, the exogenous promoter is a constitutive promoter or an inducible promoter. In some embodiments, the exogenous promoter is a cell-specific promoter.

In some embodiments, the nucleic acid encoding the second donor polypeptide is inserted into a noncoding sequence in the genome of the cell. In some embodiments, the noncoding sequence is a regulatory sequence. In some embodiments, the regulatory sequence is a 3′ untranslated region of a gene, a 5′ untranslated region of a gene, an intron, an enhancer, or a silencer. In some embodiments, the regulatory sequence is an intron. In some embodiments, the nucleic acid encoding the second donor polypeptide is a synthetic exon flanked by a slice acceptor and a splice first donor site.

In some embodiments, the first donor polypeptide is a selectable marker. In some embodiments, the vector encoding the second donor polypeptide further comprises a nucleic acid sequence encoding a selectable marker. In some embodiments, the selectable marker is a protein that confers antibiotic resistance to the cell. In some embodiments, the selectable marker is fluorescent protein.

In some embodiments, the first donor polypeptide comprises a first peptide tag. in some embodiments, the second donor polypeptide comprises a second peptide tag. In some embodiments, the second donor polypeptide is BirA.

In some embodiments, the vector comprising the nucleic acid sequence encoding the second donor polypeptide further comprises a nucleic acid sequence encoding a self-cleaving peptide, wherein the nucleic acid encoding the self-cleaving peptide is located upstream of the nucleic acid sequence encoding the second donor polypeptide. In some embodiments, the self-cleaving peptides are selected from the group consisting of P2A, E2A, F2A, and T2A.

In some embodiments, the one or more recombinase target sites are flippase recognition target (FRT) sites, and wherein the recombinase is flippase. In some embodiments, the one or more recombinase target sites are loxP sites, and wherein the recombinase is Cre recombinase.

In some embodiments, the cell is eukaryotic cell or a prokaryotic cell. In some embodiments, the cell is a stem cell. In some embodiments, the stem cell is an embryonic stem cell or an induced pluripotent stem cell. Also provided is a cell or populations of cells produced by any of the methods described herein.

Also provided are methods for making genetically modified non-human animals. The methods comprise (a) introducing an embryonic stem cell produced by any of the methods described herein into a non-human animal host embryo; and (b) gestating the host embryo in a surrogate mother to produce the genetically modified non-human animal.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-IC are schematics depicting exemplary steps in a method of generating a stable cell line according to certain embodiments of this disclosure. In this exemplary method, different antibiotic selectable markers, i.e., puromycin and hygromycin, were used. First, cells were transfected and modified by a knock-in vector (e.g., a HiUGE StableTag vector). In this example, the StableTag vector expresses a puromycin resistance gene, flanked by two FRT sites at the C terminus of a gene of interest. The strategy for incorporation of the StableTag (puromycin resistance gene and FRT sites) is shown in FIG. 1A. Next, cells were selected for puromycin resistance to generate stable cell lines with the incorporated construct at the locus of interest (FIG. 1B). A second vector (e.g., a DonorTag vector), was then transduced into the puromycin-selected cells, wherein the second vector includes a donor sequence, as well as a hygromycin resistance gene (FIG. 1B). The “X” in FIG. 1B represents a stop codon. Puromycin-resistant StableTag clones transduced with a DonorTag vector along with flippase exchanged cassettes between StableTag and DonorTag through the FRT sites via recombinase-mediated cassette exchange (RMCE). Hygromycin was then used to select successfully exchanged cells containing the newly inserted donor sequence (FIG. 1C).

FIG. 2A is a schematic showing the experimental set-up and timeline for generating a StableTag HEK293T clonal cell line according to certain embodiments of this disclosure.

FIG. 2B shows the immunostaining of StableTag HEK293T clonal cells according to certain embodiments of this disclosure.

FIGS. 3A-3C are schematics depicting exemplary steps in a method of generating a stable cell line according to certain embodiments of this disclosure. In this exemplary method, different antibiotic selectable markers, i.e., puromycin and hygromycin, were used. First, cells were transfected and modified by a knock-in vector (e.g., a HiUGE StableTag vector). In this example, the StableTag vector expresses a puromycin resistance gene, flanked by two FRT sites at the C terminus of a gene of interest. The strategy for incorporation of the StableTag (puromycin resistance gene and FRT sites) is shown in FIG. 3A. Next, cells were selected for puromycin resistance to generate stable cell lines with the incorporated construct at the locus of interest (FIG. eB). A second vector (e.g., DonorTag donor or DonorTag donor^{control}), was then transduced into the puromycin-selected cells, wherein the second vector includes a donor sequence (BirA), as well as a hygromycin resistance gene (FIG. 3B). The “X” in FIGS. 3B and 3C represents a stop codon. The DonorTag donor^{control} vector, i.e., a negative control, includes a self-cleaving peptide (P2A) such that TUBB and BirA are expressed as a bicistronic mRNA transcript that encoding tubulin and BirA. The product of this transcript is cleaved to form tubulin and soluble BirA. Puromycin-resistant StableTag clones transduced with a DonorTag vector along with flippase exchange cassettes between StableTag and DonorTag through the FRT sites via recombinase-mediated cassette exchange (RMCE). Hygromycin was then used to select successfully exchanged cells containing the newly inserted donor sequence (FIG. 3C).

FIG. 4A is a schematic showing the experimental set-up and timeline for tag exchange in StableTag cells according to certain embodiments of this disclosure.

FIG. 4B shows that soluble BirA expressing “negative” cells showed diffuse BirA-HA and streptavidin staining. These cells comprise a “negative control” construct (DonorTag^{control} shown in the top panel of FIG. 4B) that, upon integration into the cells, expresses soluble BirA. As shown in the bottom panel of FIG. 4B, TUBB-DonorTag cells showed overlap staining between TUBB-V5 and TUBB-BirA-HA as well as streptavidin, suggest microtubules and its nascent proteins are biotinylated. These cells comprise a construct that, upon integration into the cells, results in expression of a TUBB-BirA fusion protein.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

“About” is used to provide flexibility to a numerical range endpoint by providing that a given value may be “slightly above” or “slightly below” the endpoint without affecting the desired result.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The compositions and methods provided herein allow for rapid, specific insertion of a nucleic acid sequence (for example, a nucleic acid sequence encoding a first peptide tag) into a cell(s), and selection of the cell(s) containing the inserted nucleic acid sequence. In some embodiments, the nucleic acid sequence is inserted into an endogenous protein coding sequence such that a fusion protein comprising the endogenous protein and the first peptide tag is expressed by the cell. Once stable cells comprising the first peptide tag are selected, the first peptide tag is easily swapped or exchanged for a second peptide tag by transfecting the cells with a recombinase and a nucleic acid sequence encoding the second peptide tag. This exchange is referred to as Recombination-mediated-cassette exchange (RMCE). This system can be used to rapidly create isogenic cell lines with a wide variety of gene/protein fusions for diverse and flexible experimental paradigms.

### I. Methods

Provided herein are methods of generating stable cell lines. The methods comprise (a) inserting a nucleic acid sequence encoding a first donor polypeptide into the genome of a population of cells via Homology-independent Universal Genome Engineering (HiUGE), wherein the nucleic acid encoding the first donor polypeptide is flanked on each side by one or more recombinase target sites; (b) selecting cells that express the first donor polypeptide; and (c) exchanging the nucleic acid encoding the first donor polypeptide in the genome of the selected cells with a nucleic acid encoding a second donor polypeptide by contacting the selected cells with: (i) a vector comprising a nucleic acid sequence encoding the second donor polypeptide, wherein the nucleic acid encoding the second donor polypeptide is flanked on each side by the one or more recombinase target sites, and wherein the one or more recombinase target sites are in frame with the coding sequence of the second donor polypeptide; and (ii) a vector encoding a recombinase that cleaves the one or more recombination target sites inserted into the genome of the selected cells and the one or more recombinase target sites in the vector, whereby the nucleic acid encoding the first donor polypeptide is exchanged for the nucleic acid encoding the second donor polypeptide in the genome of the cells via recombination-mediated cassette exchange (RMCE).

As used herein, “stable cell line” refers to a homogenous population of cells, wherein the cells comprises a nucleic acid sequence (e.g., a gene) that is stably integrated into the genome of the cells thus allowing the passage of the integrated nucleic acid sequence to future generations of the cells.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding DNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The term “polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

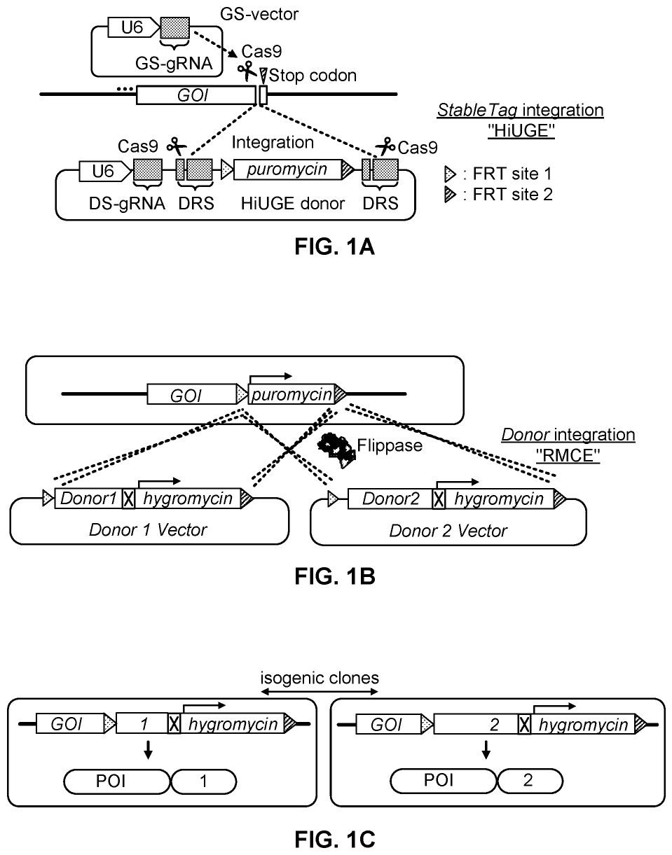

As used herein, the term “homology-independent Universal Genome Engineering (HiUGE)” or “HiUGE system” refers to a vector system that allows modification of genomic target loci, for example, modification of endogenous nucleic acid sequences encoding a polypeptide. HiUGE methods for specific insertion of a heterologous nucleic acid sequence into the genome of a cell are known. See, for example, Gao et al. “Plug and Play Modification using Homology-independent Universal Genome Engineering, Neuron 103(4): 583-597 (2019), and U.S. Patent Application Publication No. 20210047643, both of which are incorporated herein by this reference. In some embodiments, HiUGE uses a two-vector approach for higher-throughput genomic knock-in (KI) applications. The donor vector (HiUGE vector) contains an insertional DNA fragment (e.g. a payload, such as, a nucleic acid sequence encoding a first donor polypeptide, also referred to as HiUGE donor) that is flanked on both ends by an artificial DNA sequence (DRS) that is non-homologous to the target genome, wherein each DRS comprises a cleavage site for a CRISPR-based nuclease, for example, Cas9. This sequence is recognized by a donor-specific gRNA (DS-gRNA) that directs Cas9-mediated autonomous excision and release of the payload. The payload can then be integrated into the genome of the cell, via non-homologous end joining (NHEJ), into one or more endogenous genes of interest, at one or more genomic loci specified by one or more gene-specific gRNA (GS-gRNA) vector(s). See, for example, FIG. 1A.

As used throughout, the term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a single guide RNA), or micro RNA.

As used herein, the term “genome editing” or “gene editing” refers to changing or modifying the genome of a cell. Genome editing may include correcting or restoring a mutant gene, knocking out a gene, or knocking in a gene. Genome editing may also be used to introduce a label or tag onto a protein, as described in the Examples.

As used herein, the term “endogenous” with reference to a nucleic acid, for example, a gene, or a protein in a cell, is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

As used herein the phrase “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

As used herein “non-homologous end joining (NHEJ) pathway” refers to a pathway that repairs double-strand breaks in DNA by directly ligating the break ends without the need for a homologous template.

The HiUGE system includes an RNA guided endonuclease, for example, a CRISPR-based nuclease or a nucleic acid sequence encoding a CRISPR-based nuclease. In some embodiments, the nucleic acid sequence encoding a CRISPR-based nuclease is DNA. In some embodiments, the nucleic acid sequence encoding a CRISPR-based nuclease is RNA. In some embodiments, a nucleic acid sequence encoding a CRISPR-based nuclease is encoded on the HiUGE vector. In some embodiments, a nucleic acid sequence encoding a CRISPR-based nuclease is encoded on the gRNA vector.

In some methods, The HiUGE system uses an intein-mediated protein splicing system that includes a Homology-Independent Universal Genome Engineering (HiUGE) vector and a gene specific vector. The HiUGE vector includes a first polynucleotide sequence encoding at least one nucleic acid sequence insert, at least one donor recognition sequence (DRS) flanking each side of the first polynucleotide sequence, a second polynucleotide sequence encoding a HiUGE vector specific gRNA, and a third polynucleotide sequence encoding a first portion of a CRISPR-based nuclease having a first split-intein. The DRS includes a cleavage site for the CRISPR-based nuclease. The HiUGE vector specific gRNA targets the CRISPR-based nuclease to the DRS and does not target a specific sequence within the subject genome. The gene specific vector includes a fourth polynucleotide sequence encoding a second portion of a CRISPR-based nuclease having a second split-intein and a fifth polynucleotide sequence that encodes a target gene specific gRNA which targets the CRISPR-based nuclease to a target gene specific sequence within the subject genome. The first portion of the CRISPR-based nuclease having a first split-intein and the second portion of the CRISPR-based nuclease having a second split-intein can join together to form a CRISPR-based nuclease. The expression of the third polynucleotide sequence and fourth polynucleotide sequence results in the generation of a first portion of the CRISPR-based nuclease polypeptide having a first split-intein and a second portion of the CRISPR-based nuclease polypeptide having a second split-intein. The first split-intein and second split-intein come together and splice the first portion of the CRISPR-based nuclease polypeptide and the second portion of the CRISPR-based nuclease polypeptide together to form an intact CRISPR-based nuclease. Thus, a fully functional CRISPR-based nuclease can be reconstituted after intein-mediated protein splicing.

As used herein, the term “intein” refers to a segment of a protein that is able to excise itself and join the remaining portions of the protein (the exteins) with a peptide bond via protein splicing. Inteins are also known as “protein introns.” A “split intein” refers to an intein of the precursor protein that comes from two genes. Examples of inteins, including split inteins, are disclosed in U.S. Pat. Appl. Publ. No. 20150232827, which is incorporated by reference herein.

In some embodiments, the target cell which is to be gene edited expresses and includes a CRISPR-based nuclease. In some embodiments, the target cell which is to be gene edited is contacted with a CRISPR-based nuclease polypeptide, for example, Cas9.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

A CRISPR-based nuclease forms a complex with the 3′ end of a gRNA. The specificity of the CRISPR-based system depends on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the CRISPR-based nuclease can be directed to new genomic targets. The PAM sequence is located on the DNA to be cleaved and is recognized by a CRISPR-based nuclease. PAM recognition sequences of the CRISPR-based nuclease can be species specific. As used herein, the term “ribonucleoprotein” complex and the like refers to a complex between a CRISPR-based nuclease, for example, Cas9, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity,” Science 351 (6268): 84-88 (2016)).

As used herein, the term “Cas9” refers to an RNA-mediated nuclease (e.g., of bacterial or archaeal origin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p 759-771, 22 Oct. 2015) and homologs thereof. It is understood that in any of the embodiments described herein, a Cas9 nuclease can be substituted with a Cpf1 nuclease or any other guided nuclease.

Examples of suitable PAM sequences recognized by Cas9 include, but are not limited, YG (SEQ ID NO: 1), wherein Y is C or T, NGG (SEQ ID NO: 2), NGA (SEQ ID NO: 3), NGCG (SEQ ID NO: 4), NGAG (SEQ ID NO: 5), NGGNG (SEQ ID NO: 6), NNGRRT (SEQ ID NO: 7), wherein R is G or A, NNNRRT (SEQ ID NO: 8). NAAAAC (SEQ ID NO: 9), NNNNGNNT (SEQ ID NO: 10), NNAGAAW (SEQ ID NO: 11), wherein W is A or T, NNNNCNDD (SEQ ID NO: 12), wherein D is G, A, or T, or NNNNRYAC (SEQ ID NO: 13.

In some embodiments, the Cas9 endonuclease is a SpCas9 endonuclease and recognizes the PAM sequence of NGG (SEQ ID NO: 2). In some embodiments, the Cas9 endonuclease is a SpCas9 variant endonuclease and recognizes the PAM sequence of NGG (SEQ ID NO: 2). In some embodiments, the Cas9 endonuclease is a SpCas9 Cas9 VRER variant endonuclease and recognizes the PAM sequence of NGCG (SEQ ID NO: 4). In some embodiments, the Cas9 endonuclease is a SpCas9 Cas9 EQR variant endonuclease and recognizes the PAM sequence of NGAG (SEQ ID NO: 5). In some embodiments, the Cas9 endonuclease is a SpCas9 VQR variant endonuclease and recognizes the PAM sequence of NGA (SEQ ID NO: 3). In some embodiments, the Cas9 endonuclease is a SaCas9 endonuclease and recognizes the PAM sequence of NNGRRT (SEQ ID NO: 7). In some embodiments, the Cas9 endonuclease is a SaCas9 Cas9 KKH variant endonuclease and recognizes the PAM sequence of NNNRRT (SEQ ID NO: 9). In some embodiments, the Cas9 endonuclease is a St1Cas9 endonuclease and recognizes the PAM sequence of NNAGAAW (SEQ ID NO: 12). In some embodiments, the Cas9 endonuclease is a St3Cas9 endonuclease and recognizes the PAM sequence of NGGNG (SEQ ID NO: 6). In some embodiments, the Cas9 endonuclease is a chimera Sp-St3Cas9 endonuclease and recognizes the PAM sequence of NGGNG (SEQ ID NO: 6). In some embodiments, the Cas9 endonuclease is an NmCas9 endonuclease and recognizes the PAM sequence of NNNNGNNT (SEQ ID NO: 10). In some embodiments, the Cas9 endonuclease is a TdCas9 endonuclease and recognizes the PAM sequence of NAAAAC (SEQ ID NO: 9). In some embodiments, the Cas9 endonuclease is a BlatCas9 endonuclease and recognizes the PAM sequence of NNNNCNDD (SEQ ID NO: 12). In some embodiments, the Cas9 endonuclease is a CjCas9 endonuclease and recognizes the PAM sequence of NNNNRYAC (SEQ ID NO: 13). In some embodiments, the Cas9 endonuclease is an FnCas9 RHA variant endonuclease and recognizes the PAM sequence of YG (SEQ ID NO: 1).

In the methods described herein, a vector comprising the nucleic acid encoding the first donor polypeptide, a vector comprising the second donor polypeptide and/or a vector comprising a nucleic acid sequence encoding the recombinase can be introduced into one or more cells. As used herein, the phrase “introducing” in the context of introducing a nucleic acid refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

The term “vector,” as used herein, means a nucleic acid sequence containing an origin of replication. A vector may be a viral vector (for example, an adenoviral vector, a lentiviral vector or an adeno-associated (AAV) vector), bacteriophage, bacterial artificial chromosome or yeast artificial chromosome. A vector may be a DNA or RNA vector. A vector may be a self-replicating extrachromosomal vector, and optionally, is a DNA plasmid. In some embodiments, the vector may encode one or more polypeptides and/or at least one gRNA molecule.

In the methods for making stable cells lines provided herein, the nucleic acid sequence encoding the first donor polypeptide is flanked on each side by one or more recombinase target sites. As described above, the nucleic acid sequence encoding the first donor polypeptide is integrated into the genome of the cell via HiUGE. To facilitate the exchange of the genomically integrated nucleic acid encoding the first donor polypeptide with the second donor polypeptide, the nucleic acid sequence encoding the second donor polypeptide is flanked on each side by the same one or more recombinase target sites that flank the nucleic acid encoding the first donor polypeptide used in step (a) of the methods. Since the recombinase recognizes the one or more recombinase target sites integrated into the genome, as well as the one or more recombinase target sites on the nucleic acid sequence encoding the second donor polypeptide, when the nucleic acid sequence encoding the second donor polypeptide is introduced into the cell, and the cell is contacted with the recombinase, the nucleic acid sequence encoding the first donor polypeptide is exchanged with the nucleic acid sequence encoding the second donor polypeptide, thus inserting the second donor polypeptide into the genome of the cell. This method allows precise insertion of the second donor polypeptide into the specific genomic loci where the first donor polypeptide was originally inserted, without introducing off-target effects into the genome of the cell.

As used throughout, the term “recombinase” refers to an enzyme that catalyzes the exchange of DNA segments at specific recombinase target sites. The recombinases used in the methods provided herein can be delivered to a cell via an expression cassette on an appropriate vector, such as a plasmid or viral vector. In other embodiments, recombinases can be delivered to a cell as a protein, simultaneously or sequentially with the nucleic acid encoding the second donor polypeptide. In yet other embodiments, the recombinase could be encoded in the cell and expressed under the control of an inducible promoter.

In some embodiments, the recombinase is a cyclization recombination enzyme (Cre) and the recombinase target sites are lox recombination sites. Alternatively, the recombinase is flippase and the recombinase target sites are flippase recognition targets (FRTs). In some embodiments, the recombinase target sites can be mutant recombinase target sites. In some embodiments, the recombinase is DreO recombinase and the recombinase target sites are rox sites that are recognized by DreO recombinase. See, for example, Anastassiadis et al. Dis. Model Mech. 2(9-10): 508-515 (2009). Also, see Tian and Zhou “Strategies for site-specific recombination with high efficiency and precise spatiotemporal resolution,” J. Biol. Chem. 296: 100509 (2021), for additional recombination systems that can be used.

In the methods provided herein, the first donor polypeptide and the second donor polypeptide can be any polypeptide, including full-length proteins or fragments thereof. For example, and not to be limiting, the polypeptide can be an enzyme, a hormone, a structural protein, a receptor, a signal protein, a signal peptide, a peptide tag (e.g., an epitope), a transport protein, or a selectable marker, to name a few. In some embodiments, the nucleic acid encoding the second donor polypeptide is inserted into a coding sequence for a gene. In some examples, the nucleic acid encoding the second donor polypeptide is inserted into the coding sequence for a gene upstream of the stop codon for the gene. In some embodiments, the nucleic acid encoding the second donor polypeptide is inserted at the C-terminus of an endogenous protein, or at the N-terminus of an endogenous protein. In some embodiments, the cell expresses a fusion polypeptide comprising the second donor polypeptide and an endogenous polypeptide encoded by the gene. In some embodiments, the expression of the second donor polypeptide or the fusion polypeptide is under the control of the endogenous promoter for the gene. In some examples, where the expression of the second donor polypeptide, for example, a selectable marker, is under the control of the endogenous promoter (for example, in methods where the vector comprising the second donor polypeptide does not comprise a promoter), only cells with the correct insertion at a target gene will express the marker. As only cells with the correct insertion at the target gene will express the marker, this could potentially reduce background cells during the selection.

In some embodiments, the second donor polypeptide is inserted into the coding sequence of a gene to replace a domain of an endogenous protein. For example, the second donor polypeptide can be an extracellular domain, a transmembrane domain or an intracellular domain of an endogenous transmembrane protein. In this way, chimeric proteins comprising an endogenous domain(s) and heterologous domains with altered properties, for example, altered binding or intracellular signaling, can be expressed by the cell. In some embodiments, a functional property of the heterologous domain is imparted on the endogenous protein. In some embodiments, the second donor polypeptide is an enzyme or an active fragment thereof. In some embodiments, the second donor polypeptide is a targeting motif that directs an endogenous protein to a different cellular location. In some embodiments, the second donor polypeptide is a peptide sequence that targets an endogenous protein for degradation, for example, a degron sequence. In some embodiments, the second donor polypeptide is a, E. coli BirA sequence or an ancestral BirA for proximity-dependent biotin identification (AirID) sequence that is fused to an endogenous protein to identify proximal proteins that interact with the endogenous protein. See, for example, Kido et al., eLife 2020; 9:e54983, and Fairhead and Howarth, “Site-specific biotinylation of purified proteins using BirA,” Methods Mol. Biol. 1266: 171-184 (2015). In some embodiments, TurboID or UltraID is used for contact-dependent proximity labeling. See, for example, Xu and Fan, “In vivo interactome profiling by enzyme-catalyzed proximity labeling,” Cell & Bioscience 11, Article number: 27 (2021).

Using the methods described herein, once a stable population of cells comprising the first donor polypeptide is made, the nucleic acid sequence encoding the first donor polypeptide can be readily replaced with the nucleic acid sequence encoding the second donor polypeptide to rapidly generate a cell line that expresses the second donor polypeptide. Since the cell lines are stable, a population of cells derived from the original population of cells that expresses the first donor polypeptide can be used to replace the first donor polypeptide with any second donor polypeptide, thus allowing rapid generation of multiple stable cell lines that express a second donor polypeptide of interest or a fusion polypeptide comprising an endogenous protein and the second donor polypeptide of interest. For example, to ascertain the effects of targeting an endogenous protein to different cellular locations, several (for example, two or more) stable cell lines, wherein each cell line expresses an endogenous protein fused to a different targeting motif can be made using the methods described herein without having to characterize each cell line. In another example, to ascertain the effects of multiple enzymes fused to an endogenous protein, several (for example, two or more) stable cell lines, wherein each cell line expresses an endogenous protein fused to a different enzyme can be made using the methods described herein. Thus, the methods provided herein can be used to generate multiple, essentially genetically identical cell lines (i.e., isogenic cell lines).

As used herein, a “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

In some embodiments, the vector comprising the nucleic acid sequence encoding the second donor polypeptide further comprises an exogenous promoter operably linked to the nucleic acid sequence encoding the second donor polypeptide. In some embodiments, the exogenous promoter is a constitutive promoter or an inducible promoter. A “constitutive” promoter is a promoter that is active under most environmental and developmental conditions. Examples of constitutive promoters include, but are not limited to, a CMV promoter, a U6 promoter, a PGK promoter, a EF-1α promoter and a SV40 promoter.

An “inducible” promoter is a promoter that is active under environmental or developmental regulation, for example, regulated by the presence or absence of a drug. Examples of inducible promoters include, but are not limited to, the pL promoter (induced by an increase in temperature), the pBAD promoter, (induced by the addition of arabinose to the growth medium). the tetracycline-controlled transcriptional activation system (Tet-On/Tet-Off, Bujard and Gossen, PNAS, 89(12):5547-5551 (1992)), the Lac switch inducible system (Wyborski et al., Environ Mol Mutagen, 28(4):447-58 (1996)), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)).

In some embodiments, the exogenous promoter is a cell-specific or tissue-specific promoter. When using a cell- or tissue-specific promoter, expression occurs primarily, but not exclusively, in a particular cell or tissue. For example, expression can occur in at least 90%, 95%, or 99% of the targeted cell or tissue. It will be understood, however, that tissue-specific promoters may have a detectable amount of background or base activity in those tissues where they are mostly silent.

Examples of tissue-specific promoters include, but are not limited to, liver-specific promoters (e.g., APOA2, SERPINA1, CYP3A4, MIR122), pancreatic-specific promoters (e.g., insulin, insulin receptor substrate 2, pancreatic and duodenal homeobox 1, Aristaless-like homeobox 3, and pancreatic polypeptide), cardiac-specific promoters (e.g., myosin, heavy chain 6, myosin, light chain 2, troponin I type 3, natriuretic peptide precursor A, solute carrier family 8), central nervous system promoters (e.g., glial fibrillary acidic protein, internexin neuronal intermediate filament protein, Nestin, myelin-associated oligodendrocyte basic protein, myelin basic protein, tyrosin hydroxylase, and Forkhead box A2), skin-specific promoters (e.g., Filaggrin, Keratin 14 and transglutaminase 3), pluripotent and embryonic germ layer promoters (e.g., POU class 5 homeobox 1, Nanog homeobox, Nestin, and MicroRNA 122).

As used herein, a nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation

In some embodiments, the nucleic acid encoding the second donor polypeptide is inserted into noncoding sequence in the genome of the cell. In some embodiments, the noncoding sequence is a regulatory sequence. In some embodiments, the regulatory sequence is a 3′ untranslated region of a gene, a 5′ untranslated region of a gene, an intron, an enhancer, or a silencer. In some embodiments, the regulatory sequence is an intron. In some embodiments, the nucleic acid encoding the second donor polypeptide is a synthetic exon flanked by a slice acceptor and a splice first donor site.

In some embodiments, the vector encoding the first donor polypeptide encodes the first donor polypeptide and a selectable marker, and the vector encoding the second donor polypeptide encodes the second donor polypeptide and a selectable marker. In some embodiments, the first donor polypeptide is a selectable marker, and the vector encoding the second donor polypeptide encodes the second donor polypeptide and a selectable marker. In some embodiments, the vector comprises a stop codon between the nucleic acid sequence encoding the second donor polypeptide and the nucleic acid sequence encoding the selectable marker. See for example, FIG. 1B, where “X” indicates a stop codon. In some embodiments, the selectable marker is a protein that confers antibiotic resistance to the cell. These include, but are not limited to, kanamycin, spectinomycin, streptomycin, ampicillin, puromycin, hygromycin, carbenicillin, bleomycin, erythromycin, polymyxin B, tetracycline, chloramphenicol.

In some embodiments, the selectable marker is fluorescent protein. Examples of suitable fluorescent proteins include, but are not limited to, green fluorescent protein (GFP), enhanced green fluorescent protein (EGFP), Emerald, Superfolder GFP, Azami Green, mWasabi, TagGFP, TurboGFP, AcGFP, ZsGreen, T-Saphhire, BFP, EBFP, EBFP2, Azurite, mTagBFP, ECFP, ECFP, Cerulean, mTurquiose, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusabira Orange2, mOrange, mOrange2, tdTomato, dTomato-Tandem, TagRFP, TagRFP-T, DsRed, DsRed2, DsRed-Express (T1), DeRed-Monomer, mTangerine, mRuby, mApple, mStrawberry, AsRed2, mRFP2, mRFP1, JRed, mCherry, mGreenLantern, HcRed1, mRaspberry, dKeima-Tandem, HcRed-Tandem, mPlum, AQ143, and the like.

In some embodiments, the first donor polypeptide comprises a first peptide tag. In some embodiments, the second donor polypeptide comprises a second peptide tag. In the methods provided herein, a peptide tag, can be an epitope recognized by an antibody used for purification, staining, visualization, and the like. In some embodiments, the peptide tag is a V5 peptide tag. Other exemplary tags are FLAG, Myc, HA, histidine tag (HIS or HIStag), StrepTag, GFP, and GST.

In some embodiments, instead of encoding a second donor polypeptide, the vector comprising the nucleic acid sequence that is exchanged for the first donor polypeptide comprises a gene element sequence, for example, a regulatory sequence, that is inserted into the genome of the cell to regulate expression of an endogenous gene.

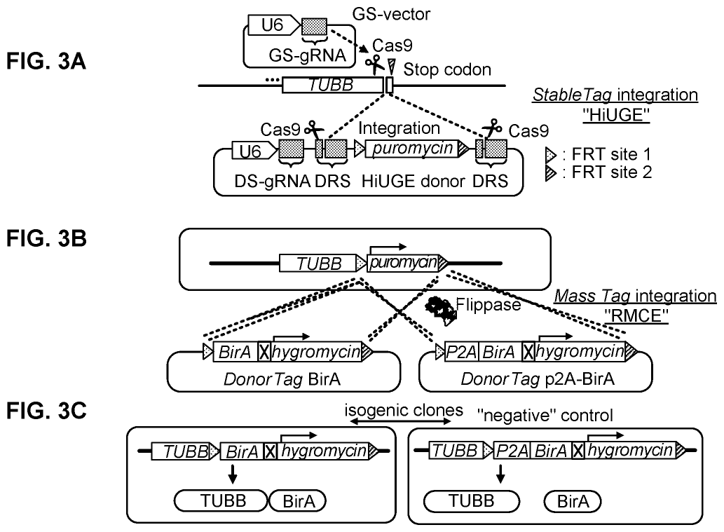

In some embodiments, the vector comprising the nucleic acid sequence encoding the second donor polypeptide further comprises a nucleic acid sequence encoding a self-cleaving peptide, wherein the nucleic acid encoding the self-cleaving peptide is located upstream of the nucleic acid sequence encoding the second donor polypeptide. By including a self-cleaving peptide, the nucleic acid encoding an endogenous polypeptide and the nucleic acid encoding the second donor polypeptide can be expressed as a bicistronic mRNA, that is then cleaved to produce the endogenous polypeptide and the second donor polypeptide. Such constructs are useful, for example, when conducting proteomic studies with BirA. For a successful proteomic analysis using BioID, it is important to set a negative control such as soluble BirA. Also, BirA fused to a protein-of-interest (POI) and soluble BirA should be performed in isogenic clones, as shown in FIG. 3C.

In some examples, two polypeptides are expressed by placing a self-cleaving peptide between a nucleic acid encoding a first polypeptide and a nucleic acid encoding a second polypeptide. Similarly, three polypeptides can be expressed multicistronically by including (a) a first self-cleaving peptide between the nucleic acid encoding the first polypeptide and a nucleic acid encoding the second polypeptide; and (b) a second self-cleaving peptide in between a nucleic acid encoding the second polypeptide and a nucleic acid encoding the third polypeptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells,” MAbs 7(2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different. In some embodiments, the self-cleaving peptides are selected from the group consisting of P2A, E2A, F2A. and T2A.

A variety of cell types can be used in the methods described herein. In some embodiments, the cells are prokaryotic cells. In other embodiments, the cells are eukaryotic cells such as, for example, plant cells, insect cells, animal cells, such as mouse, rat, hamster, non-human primate, pig, or human cells. In some embodiments, the cells are differentiating cells. In some embodiments, the cells are non-dividing cells. In some embodiments, the cell s a somatic cell. In some embodiments, the cell is a human cell.

In certain embodiments, the cell is a transformed cell. In certain embodiments, the cell is selected from the group consisting of a myoblast, a fibroblast, a glioblastoma, a carcinoma, an epithelial cell, and a stem cell. In certain embodiments, the cell is selected from the group consisting of a HEK cell, a HeLa cell, a Vero cell, a BHK cell, a MDCK cell, a NIH 3T3 cell, a Neuro-2a cell, and a CHO cell.

In some embodiments, the stem cell is an embryonic stem cell, an induced pluripotent stem cell (iPSCs) or a hematopoietic stem cell.

As used herein, the phrase “induced pluripotent stem cell” refers to a cell(s) derived from skin or blood cells that have been reprogrammed back into an embryonic-like pluripotent state that enables the development of an unlimited source of any type of human cell.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “T cell” refers to a lymphoid cell that expresses a T cell receptor molecule. T cells include human alpha beta (αφ T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4+, CD8+, or CD4+ and CD8+. T cells can also be CD4−, CD8−, or CD4− and CD8−. T cells can be helper cells, for example helper cells of type TH1, TH2, TH3, TH9, TH17, or TFH. T cells can be cytotoxic T cells. Regulatory T cells can be FOXP3+ or FOXP3−. T cells can be alpha/beta T cells or gamma/delta T cells. In some cases, the T cell is a CD4+CD25hiCD127lo regulatory T cell. In some cases, the T cell is a regulatory T cell selected from the group consisting of type 1 regulatory (Tr1), TH3, CD8+CD28−, Treg17, and Qa-1 restricted T cells, or a combination or sub-population thereof. In some cases, the T cell is a FOXP3+ T cell. In some cases, the T cell is a CD4+CD25loCD127hi effector T cell. In some cases, the T cell is a CD4+CD25loCD127hiCD45RAhiCD45RO-naïve T cell. A T cell can be a recombinant T cell that has been genetically manipulated.

In some embodiments, the cell is a primary cell. In certain embodiments, the primary cell is a neuron, a cardiomyocyte or a primary immune cell. As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

In some embodiments, the cell is derived from endoderm, ectoderm, or mesoderm. In some embodiments, the cell is eukaryotic cell. In some embodiments, the cell is a prokaryotic cell. In some embodiments, the cell is a stem cell. In some embodiments, the stem cell is an embryonic stem cell or an induced pluripotent stem cell.

Any of the methods provided herein can further comprise expanding the population of cells before or after the cells are edited. Populations of cells made by any of the methods described herein are also provided.

### II. Genetically Modified Non-Human Animals

Also provided are methods for making genetically modified non-human animals. The methods comprise (a) introducing an embryonic stem cell produced by any of the methods described herein into a non-human animal host embryo; and (b) gestating the host embryo in a surrogate mother to produce the genetically modified non-human animal.

In some methods, a genetic modification is made to a somatic cell using the methods described herein, and the nucleus of the somatic cell is transferred to an enucleated egg of the same species. In some methods, the enucleated eggs or oocytes are used for somatic cell nuclear transfer, and then transferred to a surrogate mother. In some embodiments, genetically modified zygotes resulting from somatic cell nuclear transfer are transferred to a surrogate mother.

Also provided is a genetically modified non-human animal made by any of the methods describe herein.

### III. Kits

In one aspect, provided is a kit comprising (a) a HiUge StableTag vector comprising a nucleic acid sequence encoding a first donor polypeptide, e.g., a selectable marker, wherein the nucleic acid encoding the first donor polypeptide is flanked on each side by one or more recombinase target sites; and (b) a Donor Vector comprising a cloning site for a second donor polypeptide and a nucleic acid sequence encoding a selectable marker, wherein the vector sequence comprising the cloning site and the nucleic acid sequence encoding the selectable marker nucleic is flanked on each side by the one or more recombinase target sites in the StableTag vector.

In some embodiments, the HiUge StableTag vector comprising a nucleic acid sequence encoding a first donor polypeptide, can comprise, a selectable marker and a peptide tag that can be fused onto an endogenous polypeptide. In some embodiments, the HIUge StableTag vector comprises the elements shown in FIG. 1A (i.e., a promoter, a gRNA, a first DRS comprising a Cas9 cleavage site, a recombinase target site, a nucleic acid sequence encoding a selectable marker, a second recombinase target site, and a second DRS comprising a Cas9 cleavage site). In some embodiments, the kit further comprises a gene-specific vector with a cloning site for a gene-specific gRNA. In some embodiments, the kit further comprises a vector encoding a recombinase that recognizes the one or more recombinase target sites.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of constructs including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### HiUGE Stable Tag

As shown in FIGS. 1A-C, the StableTag method relies on two rounds of selection. In this example, two different antibiotic selectable markers were used, puromycin and hygromycin, however other pairs of selectable markers, including fluorescent proteins, can be used. First, cells were transfected and modified by a knock-in vector referred to herein as the HiUGE or HiUGE StableTag vector (FIG. 1A). The HiUGE vector corresponds to the vector encoding the first donor polypeptide described throughout. The HiUGE vector expressed a puromycin resistance gene (i.e., a first donor polypeptide), flanked by two FRT sites at the C terminus of a gene of interest. The strategy for incorporation of the StableTag (puromycin resistance gene and FRT sites) is shown in (FIG. 1A). Next, cells were selected for puromycin resistance, i.e., StableTag cells, to generate stable cell lines with the incorporated construct at the locus of interest (FIG. 1B). A second vector, termed herein as DonorTag vector, is then transduced into the puromycin-selected cells, which includes a donor sequence, as well as a hygromycin resistance gene (FIG. 1B). The DonorTag vector corresponds to the vector encoding the second donor polypeptide described throughout. Multiple, different types of DonorTag vectors, each with different donor sequences for different experimental paradigms can be used in the same origin StableTag cell line. For example, different epitope tags, fluorescent proteins, enzymes, functional protein domains, or gene element sequences could be the donor in the DonorTag vectors. Puromycin-resistant StableTag clones transduced with a DonorTag vector along with flippase exchanged cassettes between StableTag and DonorTag through the FRT sites via recombinase-mediated cassette exchange (RMCE). In this example, hygromycin was then used to select successfully exchanged cells containing the newly inserted donor sequence (FIG. 1C). A key advantage of this system is that once the StableTag cell lines are made, they can be easily and efficiently modified by the cassette exchange for any donor sequence to enable a wide variety of experimental needs.

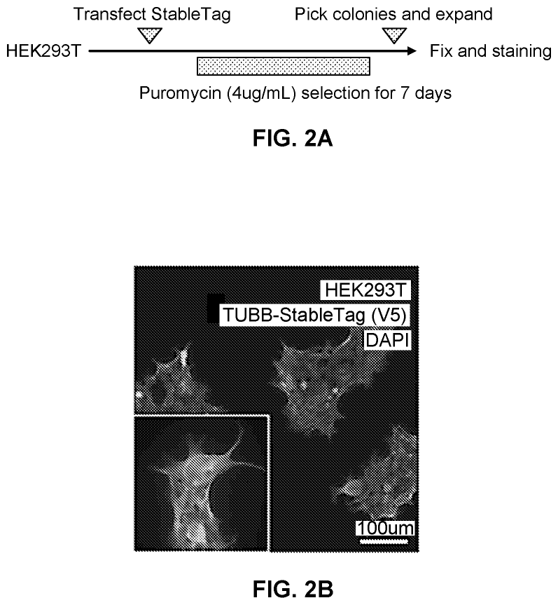

FIG. 2A shows an experimental set-up and timeline for selection-based gene tagging in HEK 293T cells according to the present disclosure. In this experiment, HEK293T cells were transfected with a StableTag vector. The StableTag vector expressed an in-frame V5 antibody epitope tag and a puromycin resistance gene, flanked by two FRT sites at the C terminus of a gene of interest. Four days later, cells were incubated with 4 μg/mL of puromycin for 7 days. Resistant cells were clonally isolated and immunostained to validate stably expressing StableTag, i.e., the V5 antibody epitope tag (FIG. 2B). As shown in FIG. 2B, tubulin (TUBB) was efficiently tagged with V5, i.e., fused with V5, upon insertion of the StableTag vector at a specific site in the coding sequence for tubulin. It is understood that the insertion can be inserted at the N-terminus of an endogenous polypeptide, at the C-terminus of an endogenous polpeptide or in an internal sequence of an endogenous polypeptide.

### EXCHANGE OF STABLETAG BY RECOMBINATION MEDIATED CASSETTE EXCHANGE (RMCE)

Experiments focused on the application of StableTag for proximity labeling were conducted. Proximity labeling is a proteomic approach to understanding protein functions based on their local interactome. BirA-dependent proximity labeling (BioID) has been used to to discover protein complexes at GABAergic and nascent excitatory synapses by overexpressing proteins fused to BirA. Yet to fully unlock the potential power of BioID, there is a great need for methods to allow for endogenous protein-BirA fusions, as overexpressed protein fusions are often associated with false positives. Because endogenous proteins are expressed at lower levels, accomplishing this goal will also require stable cell lines to generate enough material for purification and proteomic analysis. For a successful proteomic analysis using BioID, it is important to set a negative control, such as, for example, soluble BirA. Also, BirA fused to a protein-of-interest (POI) and soluble BirA should be performed in isogenic clones (FIGS. 3A-D). To accomplish these two purposes, a HiUGE based approach described herein, using cell selectable markers, and a docking site for subsequent flippase (Flp) driven RMCE of BirA fusions, was used.

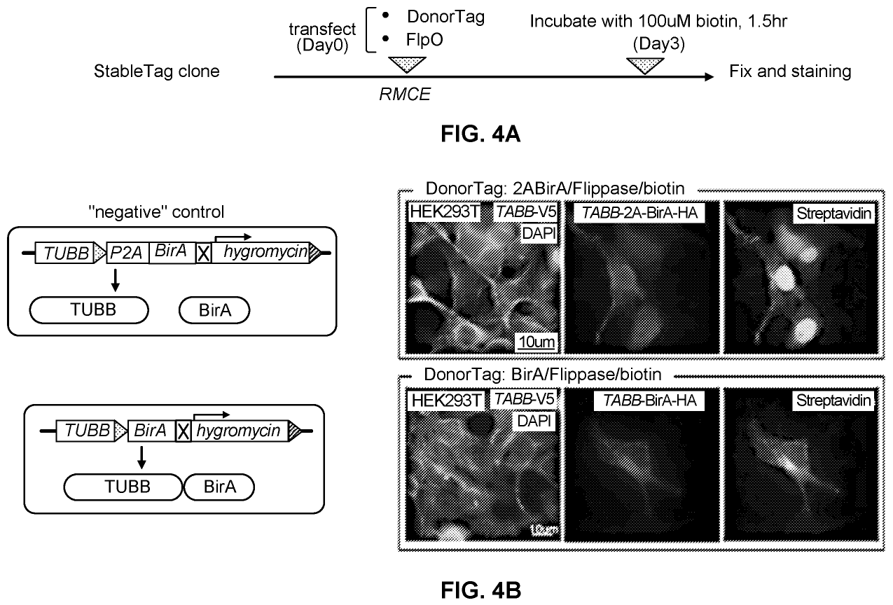

FIG. 4A provides a schematic showing the experimental set-up and timeline for tag exchange in StableTag cells. TUBB-StableTag clonal cells were transduced with a Donor Tag vector (also referred to a MassTag vector) along with Flippase (FlpO). Cells were treated with biotin for 1.5 hours followed by immunostaining with anti-HA antibody and Alexa Flour-conjugated streptavidin. As shown in the top panel of FIG. 4B, soluble BirA expressing “negative” cells (i.e., DonorTag^{control} cells) showed diffuse BirA-HA and streptavidin staining. TUBB-DonorTag cells (bottom panel of FIG. 4B) showed overlap staining between TUBB-V5 and TUBB-BirA-HA as well as streptavidin, suggesting microtubules and its nascent proteins are biotinylated.

It is understood that the examples and embodiments described herein are for illustrative purposes only and that various modifications or changes in light thereof will be suggested to persons skilled in the art and are to be included within the spirit and purview of this application and scope of the appended claims.

1. A method for generating a stable cell line comprising:
(a) inserting a nucleic acid sequence encoding a first donor polypeptide into the genome of a population of cells via Homology-independent Universal Genome Engineering (HiUGE), wherein the nucleic acid encoding the first donor polypeptide is flanked on each side by one or more recombinase target sites;
(b) selecting cells that express the first donor polypeptide; and
(c) exchanging the nucleic acid encoding the first donor polypeptide in the genome of the selected cells with a nucleic acid encoding a second donor polypeptide by contacting the selected cells with:
(i) a vector comprising a nucleic acid sequence encoding the second donor polypeptide, wherein the nucleic acid encoding the second donor polypeptide is flanked on each side by the one or more recombinase target sites, and wherein the one or more recombinase target sites are in frame with the coding sequence of the second donor polypeptide; and
(ii) a vector encoding a recombinase that cleaves the one or more recombination target sites inserted into the genome of the selected cells and the one or more recombinase target sites in the vector, whereby the nucleic acid encoding the first donor polypeptide is exchanged for the nucleic acid encoding the second donor polypeptide in the genome of the cells via recombination-mediated cassette exchange (RMCE).

2. The method of claim 1, wherein the nucleic acid encoding the second donor polypeptide is inserted into a coding sequence for a gene.

3. The method of claim 2, wherein the cell expresses a fusion polypeptide comprising the second donor polypeptide and an endogenous polypeptide encoded by the gene.

4. The method of claim 1, wherein expression of the second donor polypeptide or the fusion polypeptide is under the control of the endogenous promoter for the gene.

5. The method of claim 1, wherein the vector comprising the nucleic acid sequence encoding the second donor polypeptide further comprises an exogenous promoter operably linked to the nucleic acid sequence encoding the second donor polypeptide.

6. The method of claim 5, wherein the exogenous promoter is a constitutive promoter or an inducible promoter.

7. The method of claim 5, wherein the exogenous promoter is a cell-specific promoter.

8. The method of claim 1, wherein the nucleic acid encoding the second donor polypeptide is inserted into a noncoding sequence in the genome of the cell.

9. The method of claim 8, wherein the noncoding sequence is a regulatory sequence.

10. The method of claim 9, wherein the regulatory sequence is a 3′ untranslated region of a gene, a 5′ untranslated region of a gene, an intron, an enhancer, or a silencer.

11. The method of claim 10, wherein the regulatory sequence is an intron.

12. The method of claim 11, wherein the nucleic acid encoding the second donor polypeptide is a synthetic exon flanked by a slice acceptor and a splice first donor site.

13. The method of claim 1, wherein the first donor polypeptide is a selectable marker.

14. The method of claim 1, wherein the vector encoding the second donor polypeptide further comprises a nucleic acid sequence encoding a selectable marker.

15. The method of claim 13, wherein the selectable marker is at least one of a protein that confers antibiotic resistance to the cell or a fluorescent protein.

16. (canceled)

17. The method of claim 1, wherein the first donor polypeptide comprises a first peptide tag.

18. The method of claim 17, wherein the second donor polypeptide comprises a second peptide tag.

19. The method of claim 1, wherein the second donor polypeptide is BirA.

20. The method of claim 1, wherein the vector comprising the nucleic acid sequence encoding the second donor polypeptide further comprises a nucleic acid sequence encoding a self-cleaving peptide, wherein the nucleic acid encoding the self-cleaving peptide is located upstream of the nucleic acid sequence encoding the second donor polypeptide.

21. The method of claim 20, wherein the self-cleaving peptides are selected from the group consisting of P2A, E2A, F2A, and T2A.

22. The method of claim 1, wherein the one or more recombinase target sites are flippase recognition target (FRT) sites, and wherein the recombinase is flippase.

23. The method of claim 1, wherein the one or more recombinase target sites are loxP sites, and wherein the recombinase is Cre recombinase.

24. The method of claim 1, wherein the cell is eukaryotic cell or a prokaryotic cell.

25. A cell produced by the method of claim 1.

26. The method of claim 1, wherein the cell is a stem cell.

27. The method of claim 26, wherein the stem cell is an embryonic stem cell or an induced pluripotent stem cell.

28. A method for making genetically modified non-human animal comprising:
(a) introducing an embryonic stem cell produced by the method of claim 27 into a non-human animal host embryo; and
(b) gestating the host embryo in a surrogate mother to produce the genetically modified non-human animal.
